(19) United States
(12) Patent Application Publication
(10) Pub. No.: US 2016/0278737 A1
(43) Pub. Date: Sep. 29, 2016

(54) ULTRASOUND ENDOSCOPE

(71) Applicant: OLYMPUS CORPORATION, Tokyo (JP)

(72) Inventor: Takanao FUJIMURA, Sagamihara-shi (JP)

(73) Assignee: OLYMPUS CORPORATION, Tokyo (JP)

(21) Appl. No.: 15/177,773

(22) Filed: Jun. 9, 2016

Related U.S. Application Data
(63) Continuation of application No. PCT/JP2015/056504, filed on Mar. 5, 2015.

(30) Foreign Application Priority Data
Sep. 2, 2014 (JP) 2014-178310

(51) Int. Cl. A61B 8/12; A61B 1/005; A61B 1/00; A61B 1/015; A61B 1/06; A61B 8/00; A61B 1/018

(52) U.S. Cl. CPC A61B 8/12; A61B 8/4281; A61B 8/4483; A61B 1/0051; A61B 1/018; A61B 1/015; A61B 1/0661; A61B 1/00114; A61B 1/0008

(57) ABSTRACT

An ultrasound endoscope includes: an acoustic lens for transmitting/receiving ultrasound; a transducer element generating ultrasound vibration transmitted/received via the acoustic lens; backing material with an insulation property provided on a face of the transducer element opposite to the acoustic lens; a housing accommodating the acoustic lens, the transducer element and the backing material so as to expose a surface of the acoustic lens to an outside; an insulative cooling portion with thermal conductivity higher than thermal conductivity of the backing material, and laminated on a surface of the backing material opposite to a surface in contact with the transducer element; and a signal wire configured with a metal wire extended from the transducer element into the housing through the backing material, the signal wire, including a curved portion curved so that an area of contact with the cooling portion is increased, being covered with the cooling portion.

### CROSS REFERENCE TO RELATED APPLICATION

This application is a continuation application of PCT/JP2015/056504 filed on Mar. 5, 2015 and claims benefit of Japanese Application No. 2014-178310 filed in Japan on Sep. 2, 2014, the entire contents of which are incorporated herein by this reference.

### BACKGROUND OF THE INVENTION

1. Field of the Invention

The present invention relates to an ultrasound endoscope having an ultrasound transmitting/receiving portion.

2. Description of the Related Art

In an ultrasound endoscope, because of demands for diameter reduction of an insertion portion, sensitivity improvement, and two-dimensionalization of an ultrasound transducer and the like, miniaturization and higher output of the transducer itself are demanded. Accompanying the demands, there is a tendency of increase in heat generation of the transducer itself, and there may be a case where transducer output is restricted because of increase in scope surface temperature caused by the heat generation of the transducer.

To cope with this, U.S. Patent Application Publication No. 5,545,942 discloses a technique for taking countermeasures against heat by filling heat-absorbing material in a housing of an ultrasound probe.

### SUMMARY OF THE INVENTION

An ultrasound endoscope according to an aspect of the invention includes: an acoustic lens for transmitting/receiving ultrasound; a transducer element configured to generate ultrasound vibration transmitted/received via the acoustic lens; backing material having an insulation property that is provided on a face of the transducer element opposite to the acoustic lens; a housing configured to accommodate the acoustic lens, the transducer element and the backing material in a manner that a surface of the acoustic lens is exposed to an outside; an insulative cooling portion with thermal conductivity higher than thermal conductivity of the backing material, the insulative cooling portion being laminated on a surface of the backing material opposite to a surface in contact with the transducer element; and a signal wire configured with a metal wire extended from the transducer element into the housing through the backing material, the signal wire including a curved portion curved so that an area of contact with the cooling portion is increased, and being covered with the cooling portion.

### BRIEF DESCRIPTION OF THE DRAWINGS

FIG. 1 relates to a first embodiment of the present invention and is a whole configuration diagram of an ultrasound endoscope;

FIG. 2 relates to the first embodiment of the present invention and is an explanatory diagram showing a distal end portion of the endoscope;

FIG. 3 relates to the first embodiment of the present invention and is a cross-sectional view of an ultrasound transmitting/receiving portion;

FIG. 4 relates to the first embodiment of the present invention and is a cross-sectional view along an A-A line in FIG. 3;

FIG. 5 relates to the first embodiment of the present invention and is an explanatory diagram showing a curved portion of a signal wire;

FIG. 6 relates to the first embodiment of the present invention and is an explanatory diagram showing an example in which a heat radiating member is attached;

FIG. 7 relates to a second embodiment of the present invention and is a cross-sectional view of an ultrasound transmitting/receiving portion;

FIG. 8 relates to the second embodiment of the present invention and is a cross-sectional view along a B-B line in FIG. 7; and

FIG. 9 relates to the second embodiment of the present invention and is an explanatory diagram showing an example in which a heat radiating member is attached.

### DETAILED DESCRIPTION OF THE PREFERRED EMBODIMENTS

Embodiments of the present invention will be described below with reference to drawings.

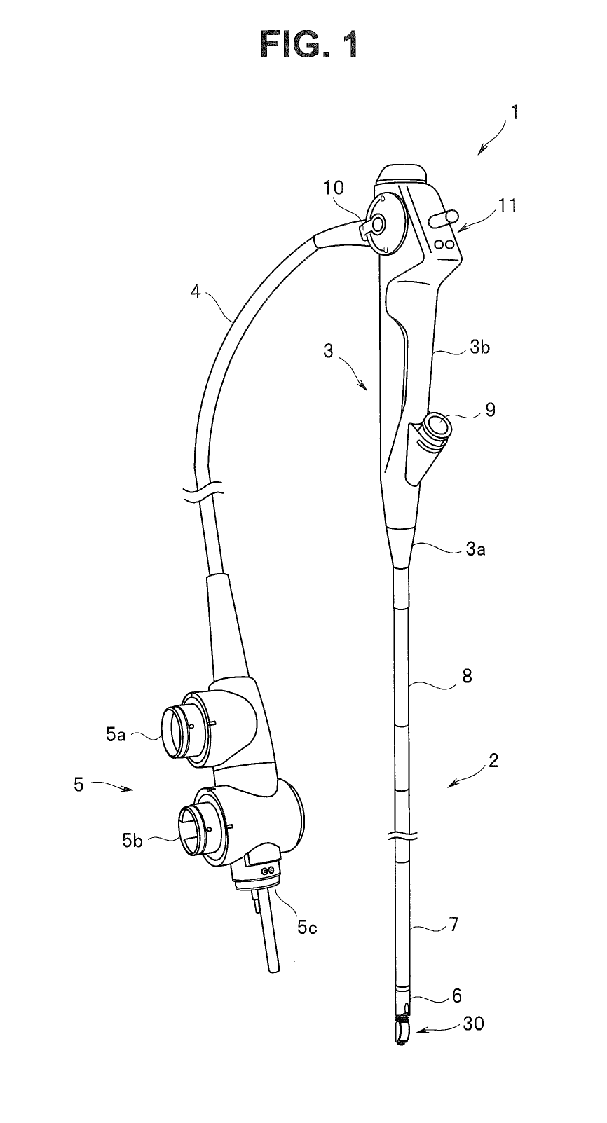

First, a first embodiment of the present invention will be described. As shown in FIG. 1, an ultrasound endoscope 1 of the present embodiment is an electronic scanning type ultrasound endoscope having an ultrasound transducer unit 30 on a distal end side of an insertion portion 2 which is formed in an elongated tube shape and is inserted into a body cavity or the like. On a proximal end side of the insertion portion 2 of the ultrasound endoscope 1, an operation portion 3 which is also used as a grasping portion is connectedly arranged. On a distal end side of a universal code 4 extended from a side portion of the operation portion 3, a connector portion 5 is arranged.

The insertion portion 2 is configured having a rigid portion 6 connectedly arranged in the ultrasound transducer unit 30 on the distal end side, a bending portion 7 connectedly arranged in a rear end side of the rigid portion 6 and configured to freely bend, for example, in an up-and-down direction, and a flexible tube portion 8 connectedly arranged in a rear end side of the bending portion 7. The flexible tube portion 8 is a long tubular member with a small diameter which is provided between the bending portion 7 and the operation portion 3 and formed to have flexibility so as to be passively flexible.

The operation portion 3 has a bend preventing portion 3a which is connected to the flexible tube portion 8, covering a proximal end of the flexible tube portion 8, and a grasping portion 3b which is connectedly arranged in the bend preventing portion 3a and which is grasped by a hand of a user when the user uses the ultrasound endoscope 1. On an upper end side of the grasping portion 3b, various kinds of operation members are arranged. On a part positioned on a lower end side of the grasping portion 3b, which is an upper part of the bend preventing portion 3a, a treatment instrument insertion port 9 for guiding a treatment instrument into the body cavity, and the like are provided. As the operation members provided on the operation portion 3, for example, a bending lever 10 for performing a bending operation of the bending portion 7, and a plurality of operation buttons 11 for performing an air/water feeding operation or a suction operation, each of operations corresponding to image pickup, illumination and the like are included.

The universal code 4 passes from a distal end of the insertion portion 2 to the operation portion 3 through insides of the bending portion 7 and the flexible tube portion 8. Furthermore, the universal code 4 is a composite cable in which various kinds of signal wires and the like extending from the operation portion 3 as well as a light guide of a light source apparatus (not shown) are inserted, and, furthermore, an air/water feeding tube extended from an air/water feeding apparatus (not shown) is inserted. The connector portion 5 arranged on the distal end side of the universal code 4 is configured having an ultrasound connector 5a for connecting to an ultrasound observation apparatus (not shown), an electrical connector portion 5b to which various kinds of signal cables are connected, and a light source side connector 5c for connecting to the light source apparatus and the air/water feeding apparatus (not shown).

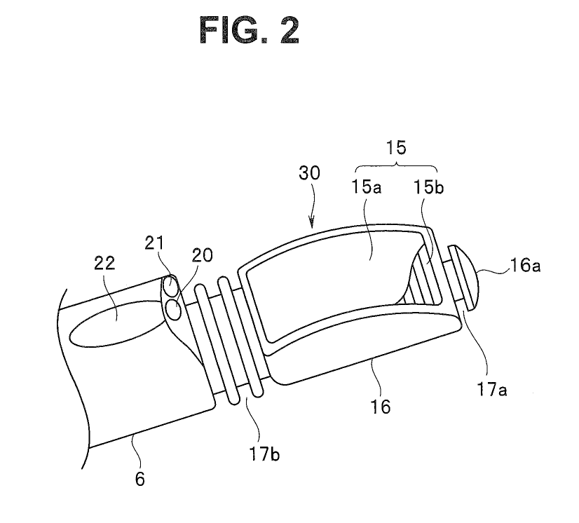

Next, a configuration of the distal end side of the insertion portion 2 will be described with use of FIG. 2. As shown in FIG. 2, the rigid portion 6 on the distal end side of the insertion portion 2 is provided with an objective lens window 20 constituting an observation optical system, an illumination lens window 21 constituting an illumination optical system, a treatment instrument guiding port 22 from which a treatment instrument such as a puncture needle is guided out, and the like.

On the other hand, the ultrasound transducer unit 30 connectedly arranged in the rigid portion 6 is configured having an ultrasound transmitting/receiving portion 15 and a nosepiece 16 which is a housing for accommodating the ultrasound transmitting/receiving portion 15. The ultrasound transmitting/receiving portion 15 is integrally arranged and held in a housing portion formed in a substantially central part of the nosepiece 16 and Ruining a recess portion. The ultrasound transmitting/receiving portion 15 is provided mainly with an acoustic lens portion 15a which forms an ultrasound transmitting/receiving surface in a longitudinal axis direction of the insertion portion 2 and a plurality of transducer elements 15b arranged along a convex surface inside the acoustic lens portion 15a.

Further, a substantially cylindrical protruding portion 16a is provided at a distal end of the nosepiece 16. A first balloon holding groove 17a is foamed on a proximal-end-side outer circumference of the protruding portion 16a, and a second balloon holding groove 17b is formed on an outer circumference of a coupling portion of the nosepiece 16 to be coupled with the rigid portion 6. For example, a thin balloon having a high contractility which is formed, for example, with silicon rubber or latex rubber is detachably interposed between the first balloon holding groove 17a and the second balloon holding groove 17b, covering the nosepiece 16.

Next, a signal wiring system of the ultrasound transducer unit 30 will be described.

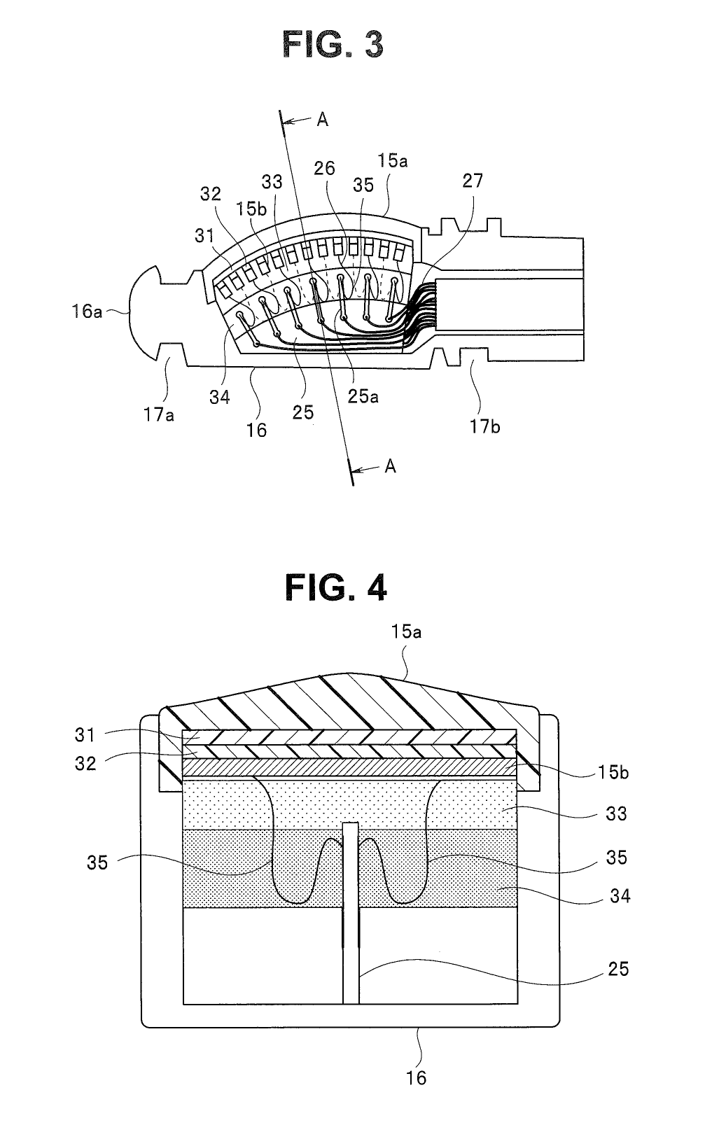

As shown in FIG. 3, the plurality of transducer elements 15b of the ultrasound transmitting/receiving portion 15 are electrically connected to a wiring substrate 25 on which corresponding signal lines are arranged as a pattern, via a plurality of signal wires 26, and the wiring substrate 25 is accommodated in the nosepiece 16. A plurality of signal cables 27 forming driving lines, the signal line and grounding lines are extended from the wiring substrate 25. The signal cables 27 are inserted through the insertion portion 2 and connected to the ultrasound connector 5a.

More specifically, in the ultrasound transducer unit 30, upper electrode sides of the transducer elements 15b are bonded to a back side of the acoustic lens portion 15a held in the substantially central portion of the nosepiece 16 via acoustic matching layers 31 and 32 for performing adjustment to obtain predetermined acoustic impedance. As the transducer element 15b, for example, a piezoelectric type element obtained by sandwiching a well-known piezoelectric element between an upper electrode and a lower electrode, or a capacitance type element obtained by separating the upper electrode and the lower electrode by a column in order to make a space with a predetermined distance between the upper electrode and the lower electrode is applicable.

On a back side of the lower electrodes of the transducer elements 15b, backing material 33 for attenuating unnecessary ultrasound is arranged. As the backing material 33, for example, what is obtained by combining ceramic particles such as alumina, zirconia and titanium oxide as filler material, with material having an insulation property, such as epoxy resin, silicone, urethane or various kinds of elastomers, as basic material can be used.

Furthermore, on a back side of the backing material 33, a cooling portion 34 for radiating heat of and cooling the transducer elements 15b is laminated. The plurality of signal wires 26 connecting the respective transducer elements 15b and the wiring substrate 25 are inserted through the backing material 33 up to the cooling portion 34, and electrically connected to the wiring substrate 25.

Metal wires the surface of which is plated with solder, tin, nickel, copper, gold or the like are used as the signal wires 26. As shown in FIGS. 3 and 4, after being curved and bent at positions inside the cooling portion 34 separated from a back side of the transducer elements 15b by a predetermined distance, the signal wires 26 are individually connected to a plurality of lands 25a of the wiring substrate 25. That is, by curving wirings of the signal wires 26 in the cooling portion 34 and forming curved portions 35, such a configuration is made that an area of contact between outer surfaces of the signal wires 26 and a member forming the cooling portion 34 is increased.

Note that, though it is assumed in FIGS. 3 and 4 that the plurality of transducer elements 15b are connected to the wiring substrate 25 via a common upper electrode, and the curved portion 35 is provided for each signal wire 26 connected to the lower electrode of each transducer element 15b and connected to the wiring substrate 25, the curved portion 35 may be provided for signal wires of both of the upper and lower electrodes.

Here, the cooling portion 34 has an insulating property and is formed with material having higher thermal conductivity than that of the backing material 33. For example, by forming the cooling portion 34 with material obtained by mixing more ceramic particles than the backing material 33 with same basic resin material as the backing material 33, radiation performance (cooling performance) is improved.

In such a wiring system of the signal wires 26 which include the curved portions 35 covered with the cooling portion 34, when each transducer element 15b is driven for transmission/reception of ultrasound, and heat is generated in each transducer element 15b, the heat is transferred to each signal wire 26. The heat transferred to the signal wire 26 is transferred to the curved portion 35 in the cooling portion 34 laminated on the back side of the backing material 33. Since the curved portion 35 has a large area of contact with a member of the cooling portion 34 having high thermal conductivity is large, the heat generated in the transducer element 15b is effectively radiated, and it is possible to efficiently emit the heat generated in the transducer element 15b to an outside.

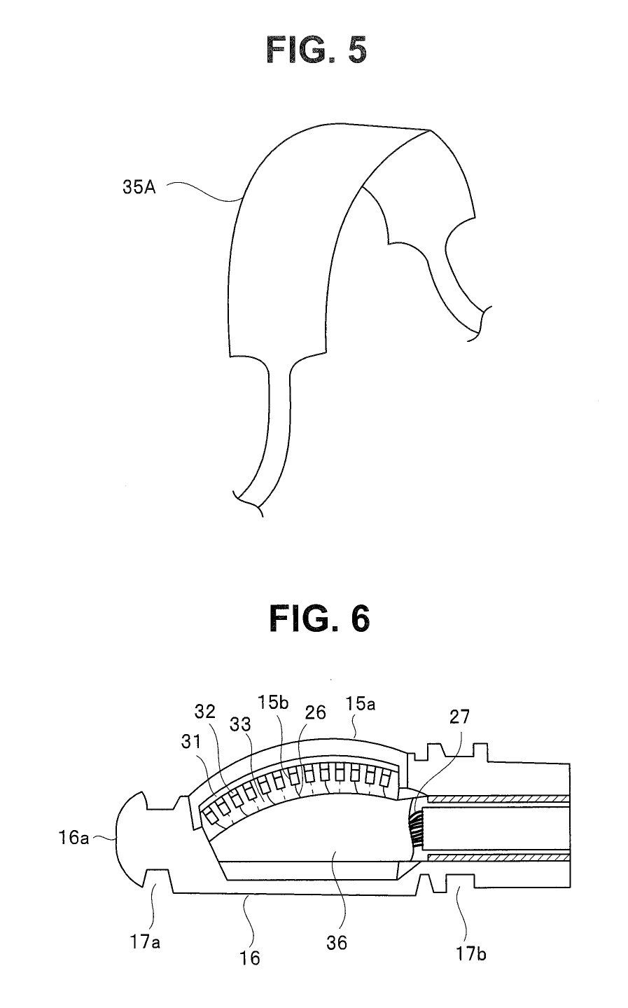

In this case, it is possible to form a part of the signal wire 26 to be arranged in the cooling portion 34, in a thin flat plate shape and curve the part in the flat plate shape to make a curved portion 35A as shown in FIG. 5. By using the curved portion 35A in the flat plate shape, it is possible to further increase the area of contact with a member constituting the cooling portion 34 and improve radiation performance more.

Further, a heat sink 36 made of metal material or the like and stuck to an outer surface of the cooling portion 34 may be arranged outside the cooling portion 34 as shown in FIG. 6. The heat sink 36 is arranged between the outer surface of the cooling portion 34 and an inner wall surface of the nosepiece 16, and one end is extended up to the rigid portion 6 on the distal end side of the insertion portion 2, so that the heat transferred from the curved portions 35 of the signal wires 26 to the member constituting the cooling portion 34 is emitted to an endoscope body side where the nosepiece 16 is connectedly arranged.

As described above, in the present embodiment, the signal wires 26 connected to the transducer elements 15b are extended into the cooling portion 34 formed with a member having high thermal conductivity on the back side of the backing material 33, and the curved portions 35 obtained by curving and bending the signal wires 26 are arranged in the cooling portion 34. Thereby, it is possible to efficiently radiate heat generated in the transducer elements 15b from the curved portions 35 with a large area of contact with the member constituting the cooling portion 34, without requiring a large space for heat radiation.

Especially in an ultrasound endoscope from which miniaturization of a distal end portion and higher output of a transducer are required, since it is possible to efficiently radiate heat of the transducer elements 15b without requiring a cooling portion with a large capacity, it is possible to suppress increase in surface temperature of the acoustic lens portion 15a and efficiently perform ultrasound observation without unnecessarily restricting output of the transducer.

Next, a second embodiment of the present invention will be described. In the second embodiment, the configuration of the cooling portion 34 in which the curved portions 35 of the signal wires 26 are arranged is changed to improve radiation performance more.

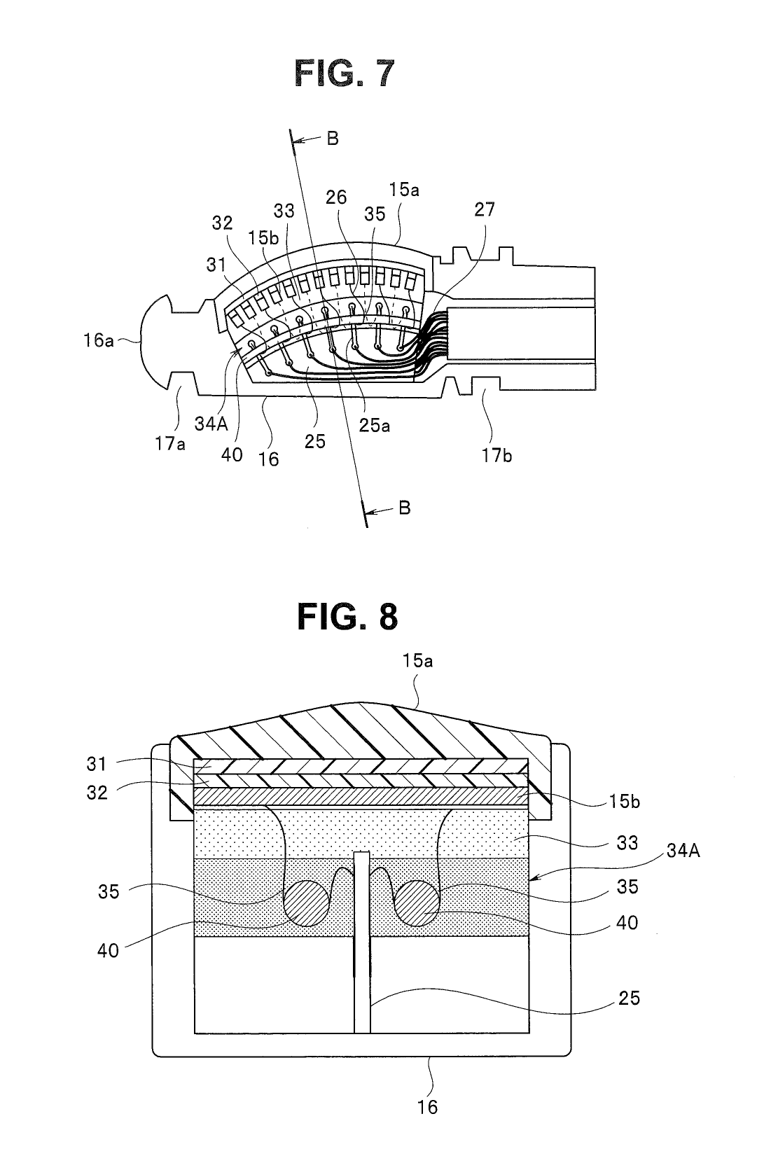

More specifically, as shown in FIG. 7, a cooling portion 34A of the second embodiment is configured, with a bar-shaped heat transfer member 40 formed with material having high thermal conductivity buried inside. An outer surface of the heat transfer member 40 with which the signal wires 26 come into contact is at least electrically insulated, and is formed with ceramic or metal material or the like with a high heat capacity. As shown in FIG. 8, the curved portions 35 of the signal wires 26 are wound and stuck around the heat transfer member 40.

Note that, in this case, for the curved portion 35, it is also possible to form a part of the signal wire 26 in a thin flat plate shape as described with regard to FIG. 5 of the first embodiment, and wind and stick the flat-plate-shaped part around the heat transfer member 40. Thereby, it is possible to increase the area of contact between the curved portion 35 and the heat transfer member 40 more and improve radiation performance more.

In such a configuration, heat generated in the transducer elements 15b is transferred through the signal wires 26, and, in the cooling portion 34A, the heat is transferred from the curved portions 35 of the signal wires 26 to the heat transfer member 40 and emitted to the outside. Since the curved portions 35 are arranged and stuck to the heat transfer member 40 having higher thermal conductivity in the cooling portion 34A, the heat from the curved portions 35 can be quickly emitted to the outside.

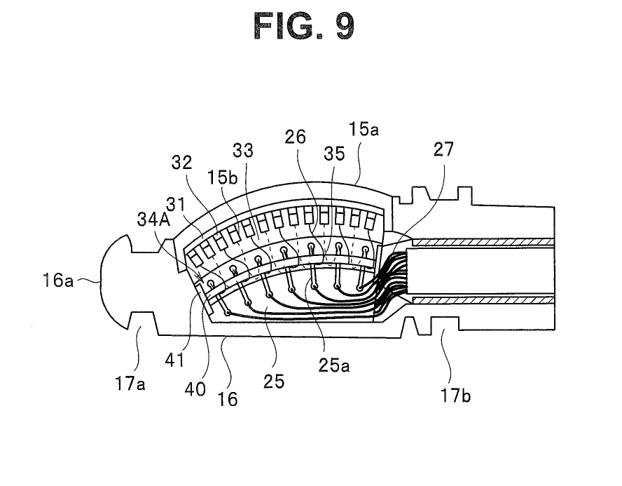

In this case also, a heat sink 41 made of metal material or the like may be arranged at an end portion of the heat transfer member 40 exposed from the cooling portion 34A, as shown in FIG. 9. The heat sink 41 is arranged along the inner wall surface of the nosepiece 16 and extended up to the rigid portion 6 on the distal end side of the insertion portion 2, similarly to the heat sink 36 described in the first embodiment, and makes it possible to quickly emit heat transferred from the curved portions 35 of the signal wires 26 to the heat transfer member 40 of the cooling portion 34A, to the endoscope body side where the nosepiece 16 is connectedly arranged.

The second embodiment makes it possible to efficiently radiate heat generated in the transducer elements 15b from the curved portions 35 of the signal wires 26 without requiring a large space for heat radiation, similarly to the first embodiment. In the second embodiment, since the curved portions 35 are arranged and stuck to the heat transfer member 40 having high thermal conductivity in the cooling portion 34A, radiation performance can be further improved.

What is claimed is:

1. An ultrasound endoscope comprising:
an acoustic lens for transmitting/receiving ultrasound;
a transducer element configured to generate ultrasound vibration transmitted/received via the acoustic lens;
backing material having an insulation property that is provided on a face of the transducer element opposite to the acoustic lens;
a housing configured to accommodate the acoustic lens, the transducer element and the backing material in a manner that a surface of the acoustic lens is exposed to an outside;
an insulative cooling portion with thermal conductivity higher than thermal conductivity of the backing material, the insulative cooling portion being laminated on a surface of the backing material opposite to a surface in contact with the transducer element; and
a signal wire configured with a metal wire extended from the transducer element into the housing through the backing material, the signal wire including a curved portion curved so that an area of contact with the cooling portion is increased, and being covered with the cooling portion.

2. The ultrasound endoscope according to claim 1, wherein a surface of the signal wire is plated.

3. The ultrasound endoscope according to claim 1, wherein the backing material is obtained by combining ceramic particles with insulative basic material as filler.

4. The ultrasound endoscope according to claim 1, wherein a part of the signal wire arranged in the cooling part is the curved portion in a flat plate shape.

5. The ultrasound endoscope according to claim 1, wherein a heat transfer member around which the curved portion is wound is buried in the cooling portion.

6. The ultrasound endoscope according to claim 1, wherein a heat sink is provided between an outer surface of the cooling portion and the housing.

7. The ultrasound endoscope according to claim 3, wherein, in the cooling portion, a larger amount of same ceramics as ceramics of the backing material is combined with same basic material as basic material of the backing material.
